(19) United States
(12) Patent Application Publication
(10) Pub. No.: US 2007/0288078 A1
(43) Pub. Date: Dec. 13, 2007

(54) APPARATUS AND METHOD FOR SKIN TIGHTENING AND CORRECTIVE FORMING

(76) Inventor: Steve Livneh, Amherstburg (CA)

Correspondence Address: HOWARD & HOWARD ATTORNEYS, P.C., THE PINEHURST OFFICE CENTER, SUITE #101, 39400 WOODWARD AVENUE, BLOOMFIELD HILLS, MI, 48304-5151, US

(21) Appl. No.: 11/687,378

(22) Filed: Mar. 16, 2007

Related U.S. Application Data
(60) Provisional application No. 60/783,777, filed on Mar. 17, 2006.
(60) Provisional application No. 60/818,702, filed on Jul. 5, 2006.

(51) Int. Cl. A61N 1/20

(52) U.S. Cl. 607/147

(57) ABSTRACT

An electrode apparatus includes a handle, a housing extending from the handle, and a plurality of wheels partially contained within the housing. A plurality of needles extend from a perimeter of each of the wheels for penetrating an epidermis of skin and into the dermis and the subcutaneous layer of skin. A contact ring is disposed adjacent the plurality of wheels and a pin interconnects the contact ring and at least one of the wheels. The plurality of wheels and the contact ring rotate in unison about a central axis. A contactor remains in continuous contact with the contact ring as the contact ring and the plurality of wheels rotate about the central axis. Electrical current, in the form of bipolar or monopolar power, is transmitted through the needles via the contactor, the contact ring, the pin, and the wheels to treat layers of the skin.

### CROSS-REFERENCE TO RELATED APPLICATIONS

The subject patent application claims priority to and all the benefits of U.S. Provisional Patent Application Ser. No. 60/783,777 which was filed on Mar. 17, 2006, the entire specification of which is expressly incorporated herein by reference and U.S. Provisional Patent Application Ser. No. 60/818,702 which was filed on Jul. 5, 2006, the entire specification of which is expressly incorporated herein by reference.

### BACKGROUND OF THE INVENTION

1. Field of the Invention

This invention relates to an apparatus and method for skin tightening. More specifically, the present invention provides an apparatus and method for introducing thermal and mechanical injury to skin via electrically charged needles.

2. Description of the Related Art

Achieving a younger looking skin having tight and well defined contours is commonly desired. Traditional devices tighten the skin by applying radiofrequency (RF) energy to the surfaced of the skin. An example of a device used to tighten the skin by applying RF energy to the surface of the skin is disclosed in U.S. patent application Ser. No. 5,755,753 to Knowlton (the '753 patent). The '753 patent discloses a device connected to an RF generator. The device includes a porous membrane which is inflated with an electrolytic solution. Once inflated, the membrane conforms to the surface of the skin. The membrane imparts a cooling effect to the skin. A plurality of electrodes are positioned at various locations in the membrane. The generator is coupled to the electrodes and a source for the electrolytic solution is coupled to the membrane. The electrodes impart radiant energy to the layers of the skin. This energy heats the skin and the underlying collagen tissue. As a result of the application of the energy to the surface of the skin, the collagen transforms its structure and contracts.

However, there exists a need for a skin tightening device which combines the benefits of applying thermal and mechanical injury.

### SUMMARY OF THE INVENTION

The present invention provides an electrode apparatus for applying electrical current to skin. The apparatus comprises a wheel defining a central hole extending along a central axis with the wheel having an outer perimeter. A plurality of needles are disposed about the perimeter and extend radially away from the central axis. An axle is disposed through the center hole along the central axis for allowing the wheel to rotate about the axle on the central axis. A contact is in electrical communication with the wheel to supply electrical current to the needles as the wheel is rolled along the skin.

Additionally, the present invention provides an electrode wheel assembly for applying electrical current to skin. The assembly comprises a first and a second plurality of wheels with each of the first and second plurality of wheels having an outer perimeter. The first and second plurality of wheels are disposed in adjacent relationship along a central axis. A plurality of needles disposed along the perimeter of each of the first and second plurality of wheels with the plurality of needles extending radially away from the central axis. The first plurality of wheels define a first connector hole extending along a first connector axis. The second plurality of wheels define a second connector hole extending along a second connector axis. The first plurality of wheels define a first bypass hole extending along the second connector axis and the second plurality of wheels define a second bypass hole extending along the first connector axis. A first pin extends along the first connector axis and through the first connector holes and the first bypass holes with the first pin in electrical communication with the first plurality of wheels. A second pin extends along the second connector axis and through the second connector holes and the second bypass holes with the second pin in electrical communication with the second plurality of wheels.

The present invention also provides a method of tightening skin using an electrode apparatus including a plurality of wheels rotatable about a central axis. Each of the plurality of wheels includes a plurality of needles disposed about each of the plurality of wheels. The method comprising the steps of placing the electrode apparatus on the skin and directing an electrical current to the plurality of needles of each of the plurality of wheels such that the needles apply the electrical current to the skin. The plurality of wheels are rotated about the central along the skin such that at least one of the plurality of needles of the wheels pierces the skin.

### BRIEF DESCRIPTION OF THE DRAWINGS

FIG. 1 is a perspective view of an electrode apparatus;

FIG. 2A is a cross-sectional view of skin illustrating needles from the electrode apparatus penetrating through an outer layer of the skin and into an epidermis layer of the skin and positive electrons flowing through the skin;

FIG. 2B is another cross-sectional view of the skin illustrating the needles from the electrode apparatus penetrating through the outer layer of the skin and into the epidermis layer of the skin and positive and negative electrons flowing into and out of the skin;

FIG. 2C is another cross-sectional view of the skin illustrating the needles from the electrode apparatus penetrating through the outer layer of the skin and into the epidermis layer of the skin and positive and negative electrons flowing into and out of the skin;

FIG. 3 is a partial perspective view of the apparatus illustrating needles from the apparatus penetrating the skin as wheels of the apparatus are rolled along the skin with coolant flowing from the apparatus an onto the skin with a monopolar application;

FIGS. 4A-4C are side views of various wheels used in an electrode wheel assembly of the apparatus;

FIG. 5 is a partial side view of the wheel used in the electrode wheel assembly;

FIG. 6 is an exploded perspective view of the apparatus for the monopolar application showing the electrode wheel assembly;

FIG. 7 is an exploded perspective view of the electrode wheel assembly for the monopolar application;

FIG. 8 is perspective view of the electrode wheel assembly;

FIG. 9 is a cross-sectional side view of the electrode wheel assembly of FIG. 8 taken along line 9-9;

FIG. 10 is a cross-sectional side view of the electrode wheel assembly of FIG. 8 taken along line 10-10;

FIG. 11 is a cross-sectional side view of the electrode wheel assembly of FIG. 8 taken along line 11-11;

FIG. 12 is a partial perspective view of the apparatus illustrating needles from the apparatus penetrating the skin as wheels of the apparatus are rolled along the skin with coolant flowing from the apparatus an onto the skin with a bipolar application;

FIG. 13 is an exploded perspective view of the apparatus for the bipolar application showing the electrode wheel assembly; and

FIG. 14 is a cross-sectional side view of the electrode wheel assembly for the bipolar application.

### DETAILED DESCRIPTION OF THE INVENTION

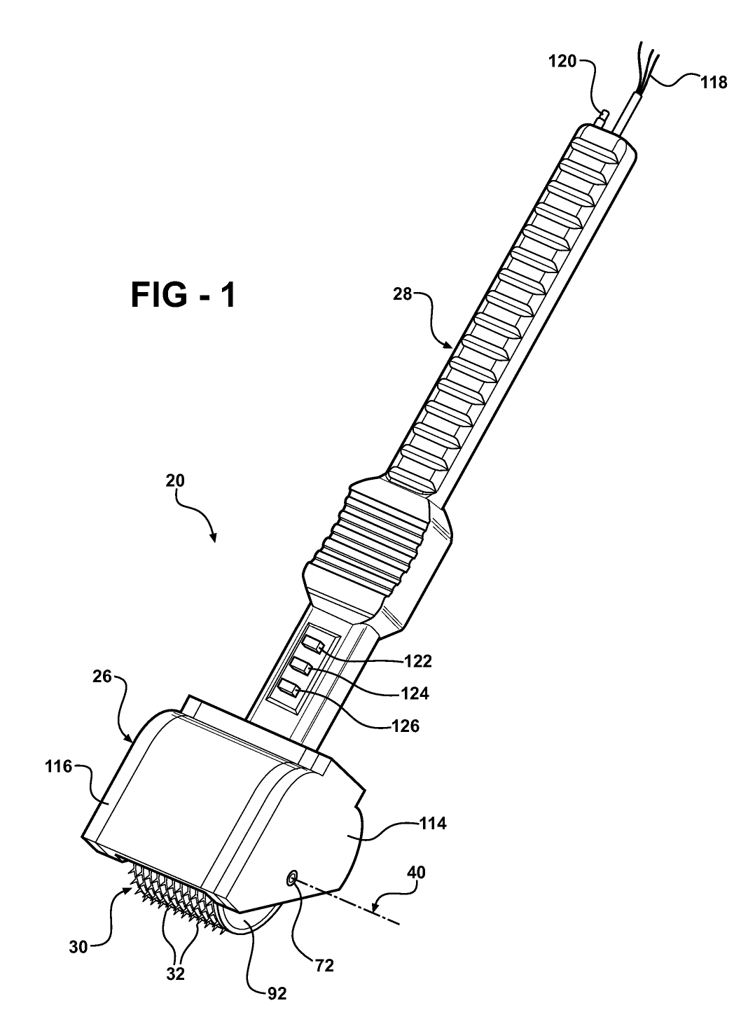

Referring to the Figures, wherein like numerals indicate corresponding parts throughout the several views, an electrode apparatus 20 is generally shown at 20 in FIG. 1. The electrode apparatus 20 is for applying electrical current to skin 22 using needles 24, i.e., electrodes, which enter the skin 22. The electrode apparatus 20 includes a housing 26, a handle 28 extending from the housing 26, and an electrode wheel assembly 30 partially contained within the housing 26.

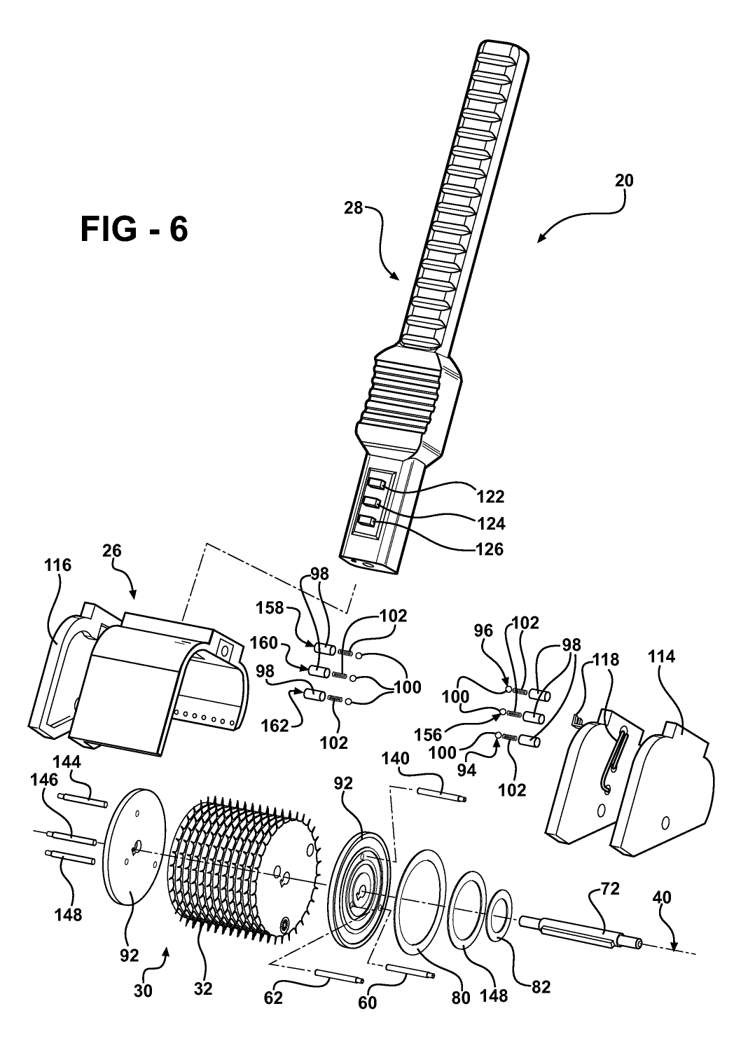

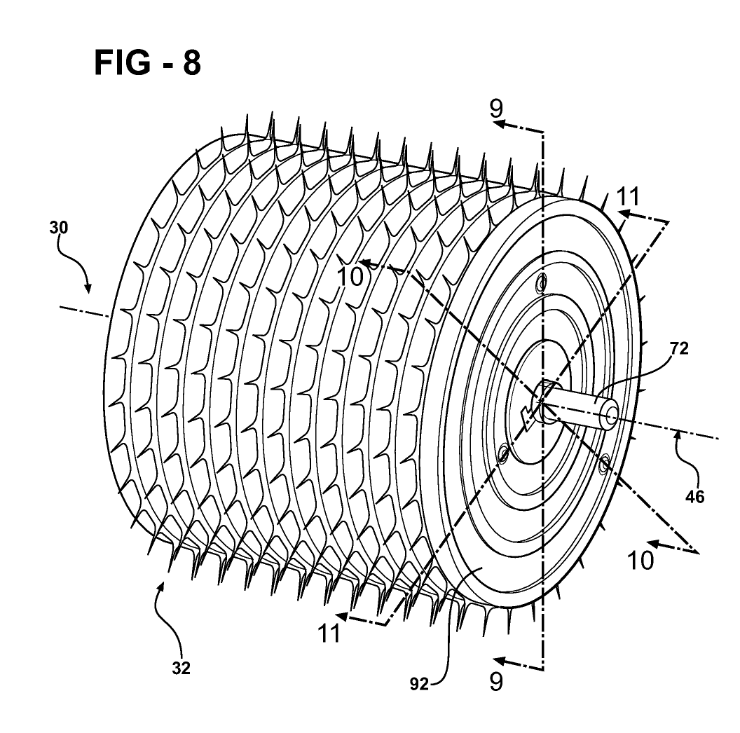

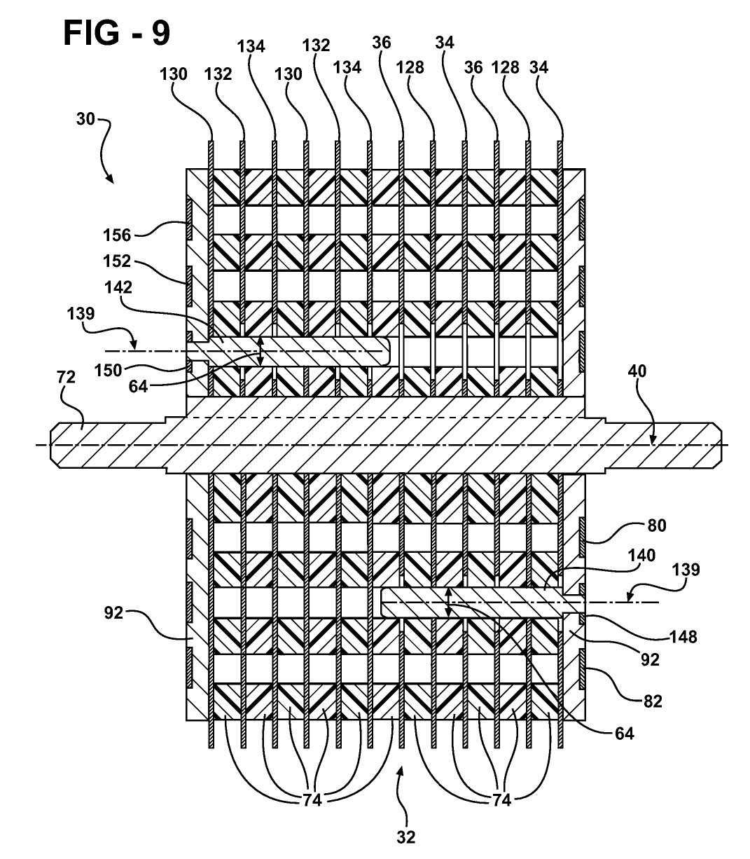

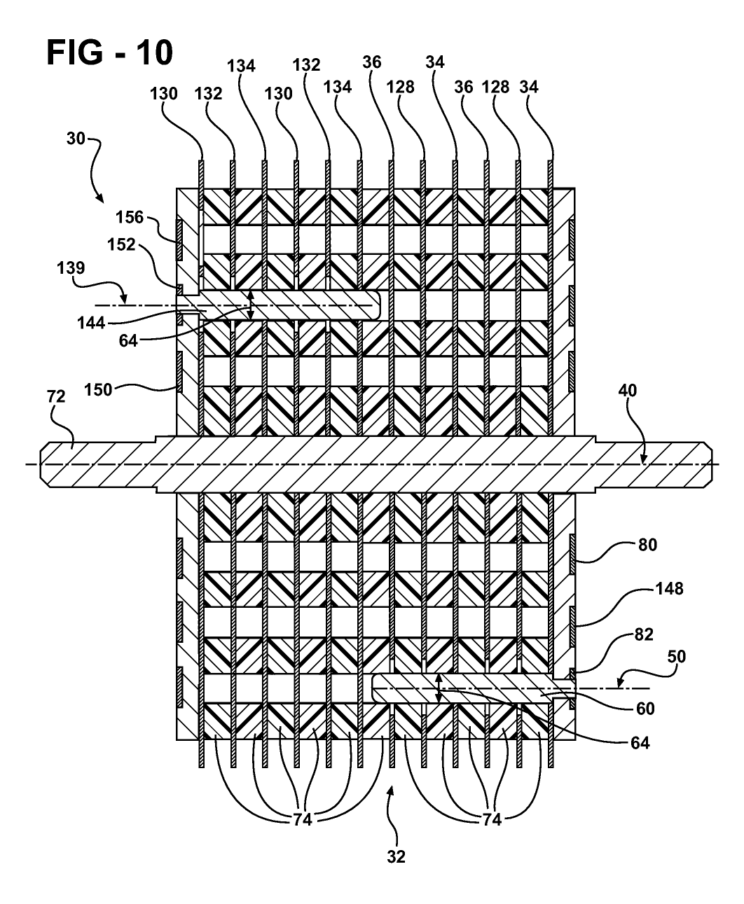

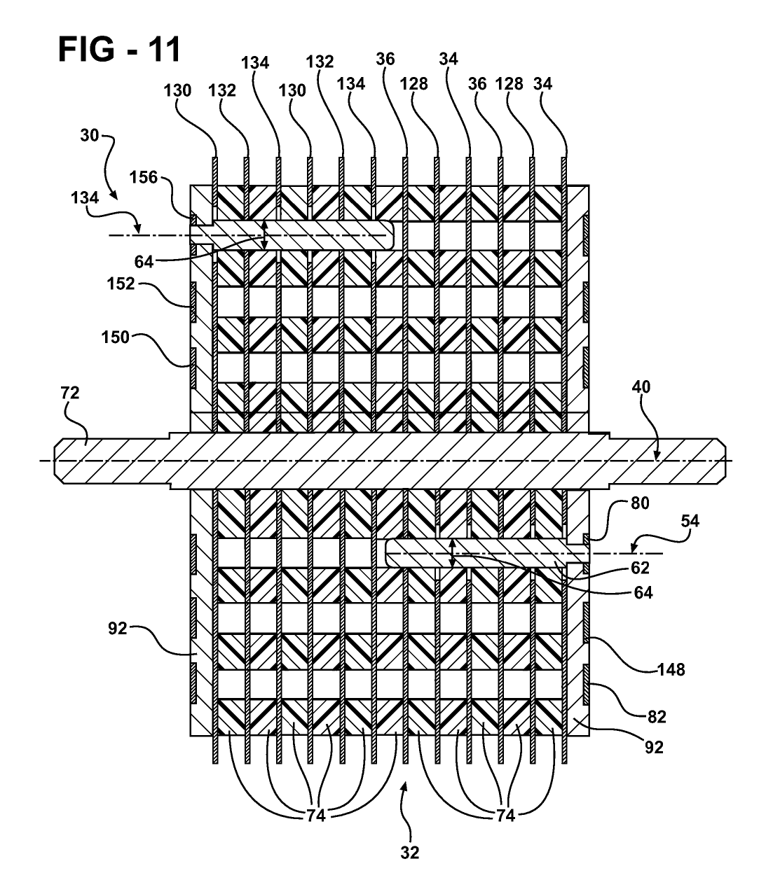

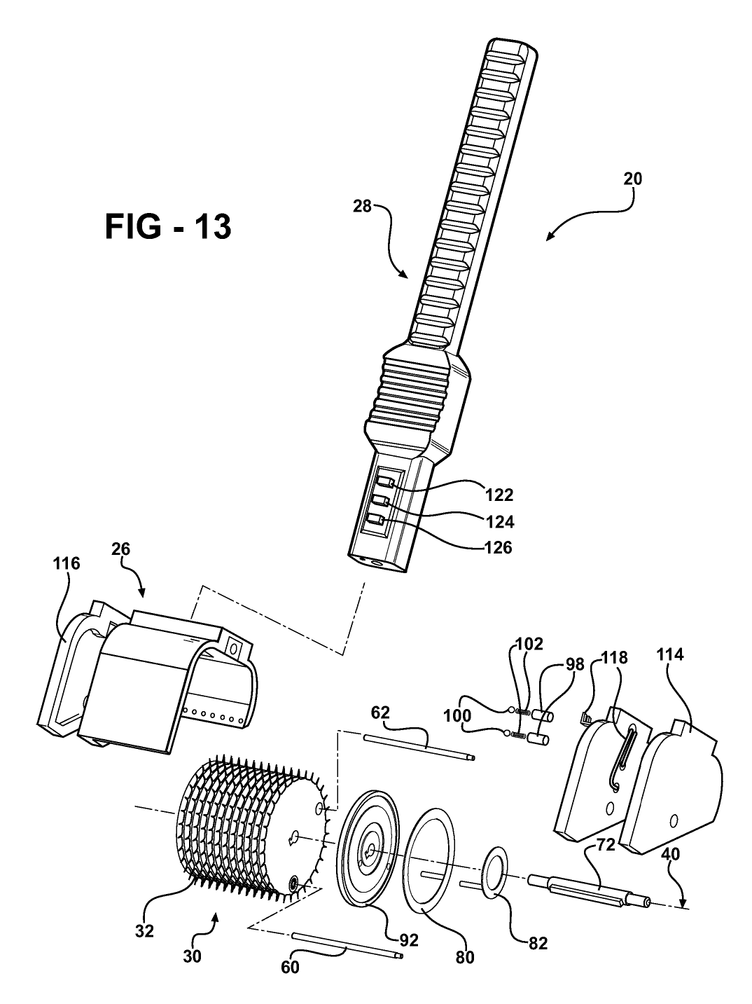

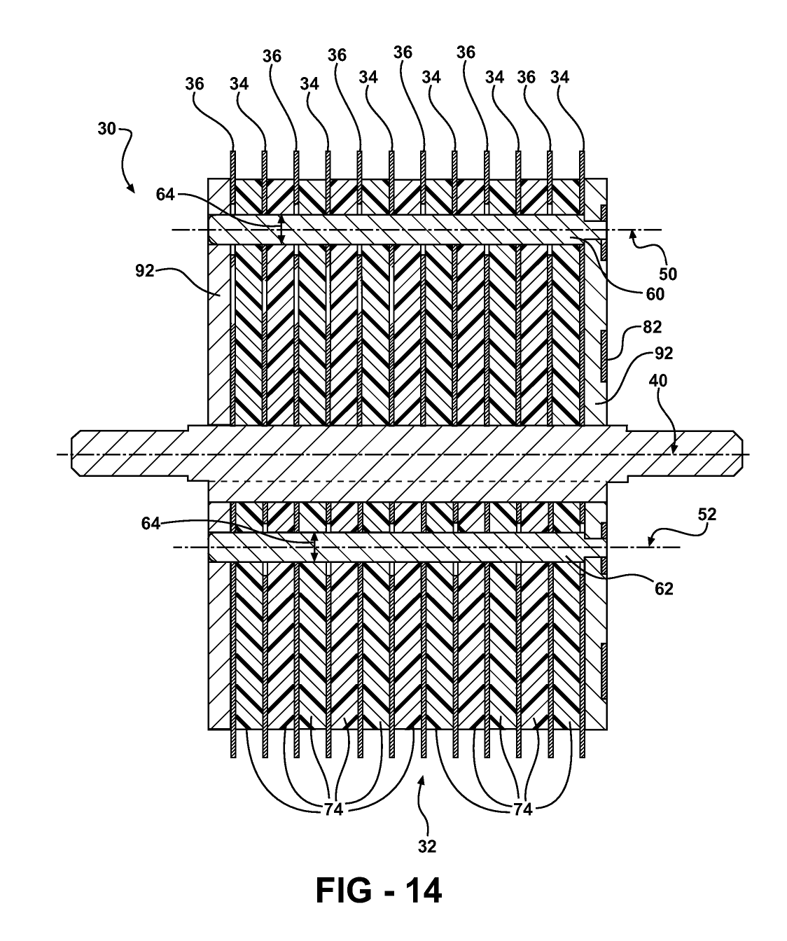

The electrode wheel assembly 30 typically includes at least two wheels 32, i.e., a first and a second wheel 36. However, more or less wheels 32 may also be used, depending on the application of the apparatus 20. For example, in one embodiment of the apparatus 20, the electrode wheels 32 assembly 30 includes twelve wheels 32, as shown generally in FIGS. 6-11 and 12-15. Each of the wheels 32 has a circular shape and includes an outer perimeter 38. The wheels 32 are rotatable about a central axis 40. The wheels 32 are formed from a conductive material, preferably stainless steel. However, the wheels 32 may also be formed from any other suitable conductive material known to those skilled in the art. The wheels 32 range typically range in diameter from 10-50 mm and a width of anywhere from 1-50 mm, depending on the specific application of the apparatus 20 and the areas of the skin 22 to be treated. However, it should be appreciated that the dimensions of the wheels 32 are not limited to these ranges, but may be any other dimension which is known to those skilled in the art. The first and second wheels 34, 36 are disposed in adjacent relationship along the central axis 40.

Each wheel is formed so that its perimeter 38 defines a plurality of the needles 24 which are sharp circumferential extensions. Each of the needles 24 extends radially away from the central axis 40. Therefore, the wheels 32 are formed such that the needles 24 extend radially when the wheels 32 are disposed concentrically along the central axis 40. The needles 24 extend from the perimeter 38 between 0.5-3 mm to a point. Preferably, the needles 24 extend 1.5-2 mm from the perimeter 38.

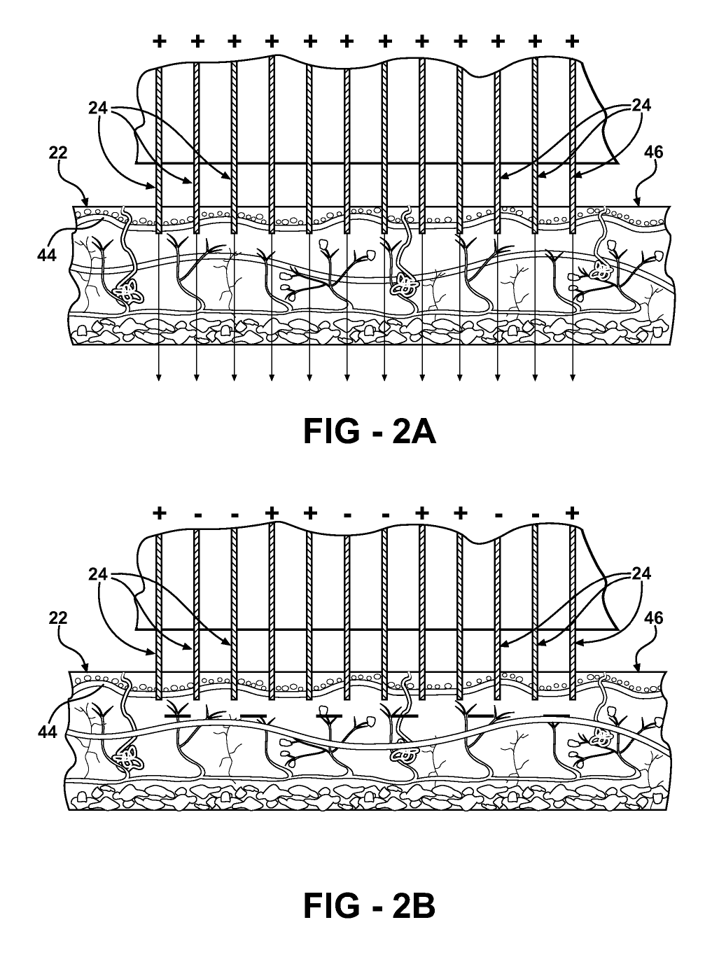

Referring generally to FIGS. 2A-2C, the electrode wheel assembly 30 is pressed onto the skin 22 by applying a force. As the electrode wheel assembly 30 is pressed onto the skin 22, at least one of the needles 24 from each of the wheels 32 pierces the skin 22, extending into the epidermis 44 of the skin 22. The wheels 32 are rotated about the central axis 40 as they move, or are rolled along and into the skin 22, such at least one of the needles 24 of each of the wheels 32 pierces the skin 22. Electrical current is supplied to the needles 24, via the wheels 32, to cause a thermal injury to the epidermis 44 and other subcutaneous layer of the skin 22. By puncturing an outer layer 46 of the skin 22, the electrical current causes a thermal injury to the epidermal layer of the skin 22 and/or other subcutaneous layers of the skin 22. After the procedure, as the epidermal layer or other subcutaneous layers of the skin 22 heal, this results in shortening and thickening of the collagen, which leads to tightening of the skin 22. Puncturing scar tissue mechanically by needles 24 results in softening of a scar and less pronounced appearance as a result of mechanical injury like punctures by the needles 24. Therefore, the apparatus 20 simultaneously induces both mechanical and thermal injury to the skin 22 due to heat build up by the application of a high frequency current, i.e., radio frequency to the skin 22 via the needles 24.

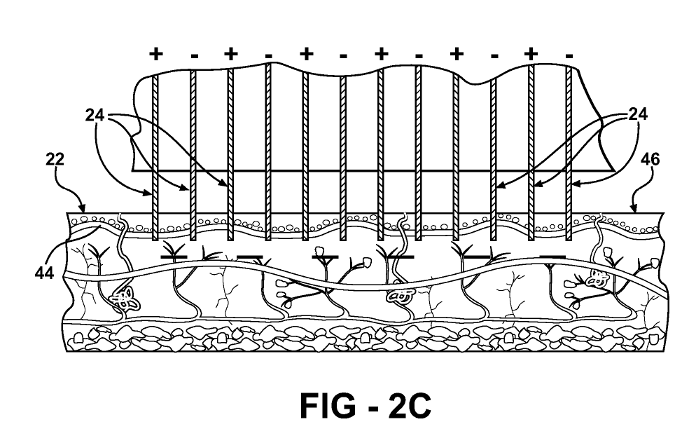

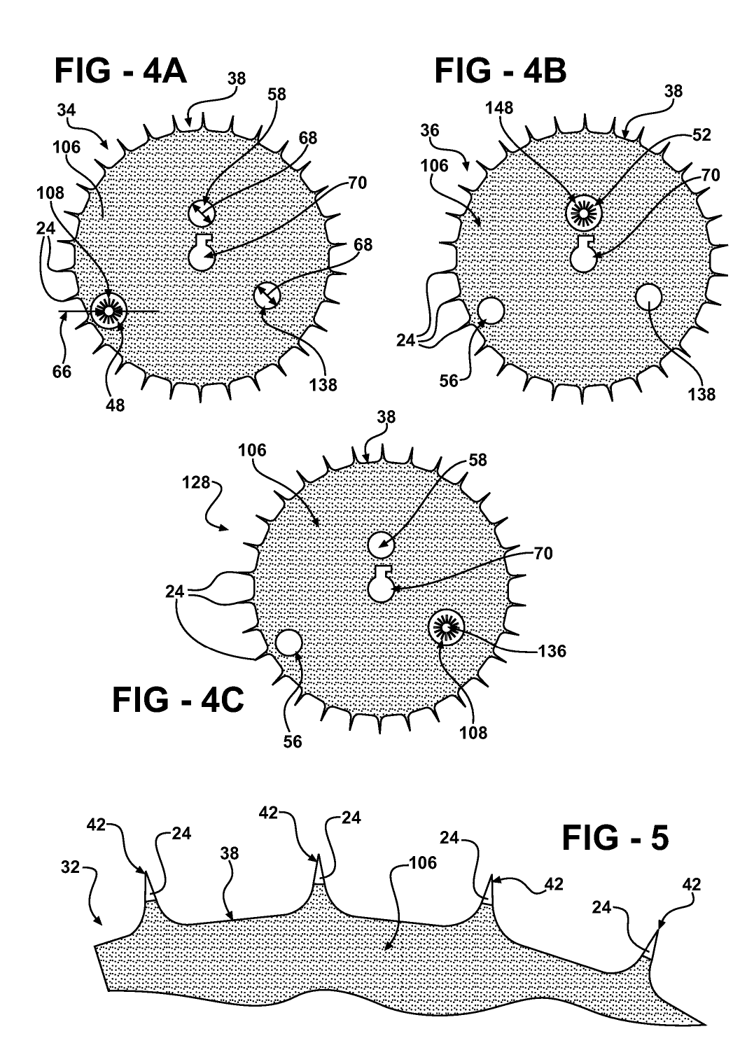

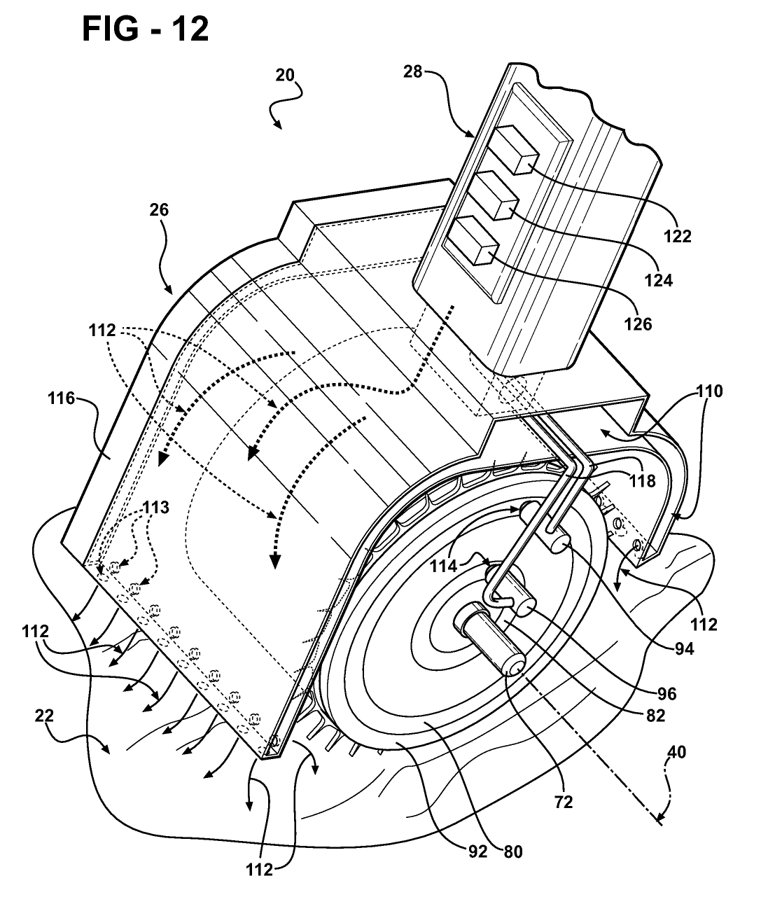

The apparatus 20 is powered either by a monopolar radio frequency or bipolar radio frequency energy. In the case of the monopolar application, the patient is attached to a ground pad (not shown) to allow current to flow through the skin 22 to the ground pad. In the case of the bipolar application, the current flows from a positively charged wheel back to a generator (not shown) via the negative wheel as will be described in more detail below. The electrical current is supplied in the form of a monopolar electrical arrangement, as shown in FIGS. 3-11, or a bipolar arrangement, as shown in FIGS. 12-15. In the monopolar arrangement, the ground pad is attached to the patient, possibly opposite the placement of the apparatus 20 during the procedure, and current is directed from a generator into the skin 22 and out to the ground pad, as illustrated in FIG. 2A. In the bipolar arrangement, the generator supplies current to one or more of the wheels 32 an into the skin 22 and back to one or more of the other wheels 32 and back to the generator, as illustrated in FIGS. 2B and 2C.

The first wheels 34 define a first connector hole 48 extending along a first connector axis 50 and the second wheels 36 define a second connector hole 52 extending along a second connector axis 54. Each of the first wheels 34 defines a first bypass hole 56 extending along the second connector axis 54. Each of the second wheels 36 defines a second bypass hole 58 extending along the first connector axis 50. A first pin 60 extends along the first connector axis 50 and through the first connector holes 48 and the first bypass holes 56 with the first pin 60 in electrical communication with the first plurality of wheels 32. A second pin 62 extends along the second connector axis 54 and through the second connector holes 52 and the second bypass holes 58 with the second pin 62 in electrical communication with the second plurality of wheels 32. The first and second pins 62 each have a rod diameter 64. The first and second connector holes 52 each have a connector diameter 66 and the first and second bypass holes 58 each have a bypass diameter 68. The bypass diameter 68 is greater than the rod diameter 64 and the rod diameter 64 is at least equal to the connector diameter 66. This means that the first pin 60 does not contact the first wheels 34 as the first pin 60 extends through the first bypass holes 56 and the second pin 62 does not contact the second wheels 36 as the second pin 62 extends through the second bypass holes 58. The first pin 60 is engaged by the first wheels 34 at the first rod hole as the first pin 60 extends through the first rod hole and the second pin 62 is engaged by the second wheels 36 at the second rod hole as the second pin 62 extends through the second rod hole. Therefore, the first pin 60 is in electrical communication with the first wheels 34 by virtue of the first pin 60 being engaged by each of the first wheels 34 and the second pin 62 is in electrical communication with the second wheels 36 by virtue of the second pin 62 being engaged by each of the second wheels 36. The first pin 60 is electrically insulated from each of the second wheels 36 as the first pin 60 extends through each of the first bypass holes 56 and the second pin 62 is electrically insulated from each of the first wheels 34 as the second pin 62 extends through each of the second bypass holes 58. Having the first wheel 34 only in electrical communication with the first pin 60 and the second wheels 36 32 only in electrical communication with the second pin 62 has many operating uses and advantages which will be discussed in more detail below.

The wheels 32 also define a central hole 70 extending along the central axis 40. An axle 72 extends through the central holes 70 along the central axis 40. The central hole 70 has a keyhole shape and the axle 72 has a shape which is complimentary to the keyhole shape of the central hole 70. Therefore, when the axle 72 is disposed through the central hole 70, the wheels 32 are locked to the axle 72 such that the wheels 32 and the axle 72 rotate about the central axis 40 in unison.

The assembly 30 also includes a plurality of insulators 74. An insulator 74 is disposed between each of the adjacent wheels 32. The insulators 74 are formed from an insulating material, such as a thermoplastic or any other suitable insulating material, for electrically insulating each wheel from the adjacent wheels 32.

The wheels 32 are arranged along the central axis 40 in a staggered configuration. This means that the wheels 32 are arranged such that the first wheels 34 alternate with the first wheels 36 along the central axis 40 such that the first wheels 34 are disposed adjacent the first wheels 36. As another staggered configuration, the wheels 32 are arranged such that two first wheels 34 are adjacent one another and two first wheels 36 are adjacent one another with the pair of first wheels 34 disposed adjacent the pair of first wheels 36. However, it is not required that the first and second wheels 34, 36 be arranged in a staggered configuration, in this non-staggered configuration all of the first wheels 34 are arranged adjacent one another as a group and all of the first wheels 36 are arranged adjacent one another as a group with the group of first wheels 34 arranged adjacent the group of first wheels 36. However, it should be appreciated that the configuration of the first and second wheels 34, 36 are not limited to these and may be any other configuration known to those skilled in the art.

A first contact 94 is in electrical communication with the first pin 60 and a second contact 96 is in electrical communication with the second pin 62 such that the first contact 94 supplies a first electrical current between only the generator and the first wheels 34 and the second contact 96 supplies a second electrical current between only the generator and the first wheels 36.

A first contact ring 80 extends from the first pin 60 and a second contact ring 82 extends from the second pin 62. Each contact ring defines a ring opening 86 with the axle 72 disposed through the ring opening 86 along the central axis 40. The first contact ring 80 has a first diameter and the second contact ring 82 has a second diameter which is larger than the first diameter such that the second contact ring 82 surrounds the first contact ring 80 about the contact axis. Each of the first and contact rings define the connector hole extending along a respective first and second connector axis 54. The first contact ring 80 is in electrical communication with the first contact 94 and rotatable about the central axis 40. The first wheels 36 and the second contact ring 82 are in electrical communication with the second contact 96. A contact plate 92 is disposed along the central axis 40 and adjacent an end of the first and second plurality of wheels 32. The first and second contact rings 82 are preferably disposed on the contact plate 92. The first contact ring 80 is disposed on the contact plate 92. The contact rings are attached to the contact plate 92 with an adhesive or friction welding. However, it should be appreciated that other forms of attachment known to those skilled in the art may also be used. The second contact ring 82 is disposed on the contact plate 92. Alternatively, there is a first and a second contact plate 92 disposed along the central axis 40. The first contact plate 92 is disposed adjacent a first end of the wheels 32 and the second contact plate 92 is disposed adjacent a second end of the wheels 32, opposed the first end of the wheels 32.

A first contact is in continuous contact with the first connector ring as the first contact ring 80 rotates about the central axis 40. A second contact 96 is in continuous contact with the second contact ring 82 as the second contact ring 82 rotates about the central axis 40. The contacts 94, 96 are typically a ball plunger including a casing 98 and a roller 100 extending from the casing 98 with a spring 102, or other biasing device, disposed within the casing 98 between the casing 98 and the roller 100. The roller 100 is typically a sphere 100. The sphere 100 has a crest 104 which remains in continuous contact with the respective first and second connector ring as the first or second contact ring 82 rotates about the central axis 40 and the sphere 100 rolls along the respective contact ring. The sphere 100 is formed from an electrically conductive material, such as a metal. The casing 98 and the spring 102 are also typically formed from an electrically conductive material. It is important that the contacts conduct an electrical current because the contacts are in continuous electrical communication with the respective contacts and the respective contact rings.

The first and second pins 62 are disposed through the respective first and second connector holes 52 to interconnect the first and second contact rings 82 to the first and second wheels 34, 36 such that the first and second wheels 34, 36 and the first and second contact rings 82 rotate about the central axis 40 in unison. The first and second contacts 94, 96 are in electrical communication with the respective first and second contact ring 82 such that electrical current is supplied to the needles 24 via the first and second wheels 34, 36, the first and second pins 62, and the first and second contact ring 82.

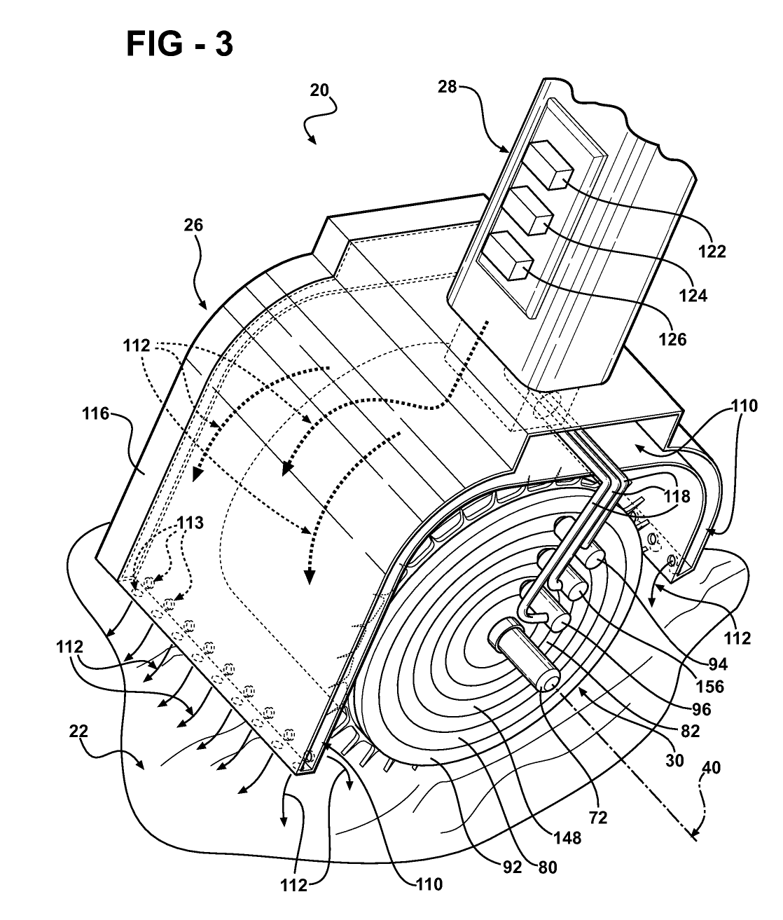

Referring to FIGS. 4A-4C, each of the wheels 32 further includes a coating 106 of dielectric material. Typically the coating 106 is suitable for at least 1000 V. The coating 106 substantially covers the wheels 32 and defines a void 108 on the wheels 32 where the coating 106 is not present. The void 108 is defined as an area which surrounds the first and second connector holes 52. The void 108 allows electrical contact the pins and the respective connector holes to allow electrical current to be supplied the needles 24. The needles 24 are at least partially free of the coating 106. Preferably, only 0.5 to 2 mm of the points 42 of the needles 24 are free of the coating 106 for allowing the electrical current to pass to/from the points 42 of the needles 24 and into or out of the skin 22.

The housing 26 is formed of a non-conductive material, such as a thermoplastic or any other suitable material known to those skilled in the art. The axle 72 is engaged within the housing 26. The housing 26 is for at least partially enclosing the wheels 32 and allowing the wheels 32 to rotate with respect to the housing 26. The housing 26 defines at least one tunnel to transport coolant 112 through the tunnel and onto the skin 22. This keeps the skin 22 cooled while performing the procedure. The housing 26 includes a first and second cover to allow the passage of wires 118 and coolant 112 therethrough. The coolant 112 is passed to a plurality of holes 113 in fluid communication with the tunnel, thus cooling the epidermis 44 prior to and after the electrode wheel passing over the skin 22. Additionally, the contacts are disposed on least one of the first and second covers 114, 116 of the housing 26. This facilitates the contacts maintain continuous contact between wires 118 and the contact rings such that all of the wheels 32 feeding current from the generator to the wheels 32 and all of the wheels 32 feeding current from the wheels 32 back to the generator are connected to separate wires 118.

The handle 28 is for being grasped by the user while performing the procedure. The handle 28 is formed such that wires 118 and a tube 120 extend through the handle 28. The wires 118 are for supplying current to/from the wheels 32 to/from the generator. There may be anywhere from one or more wires 118 passing through the handle 28. For example, in a monopolar application having twelve wheels 32 with six different wheel configurations, six positive wires 118 extend through the handle 28. In another example, in a bipolar application having twelve wheels 32 with two different wheel configurations, a positive and a negative wire 118 extend through the handle 28. The tube 120 is for transporting the coolant 112 to the housing 26 tunnel of the housing 26. The tube 120 is also in fluid communication with a coolant 112 source (not shown), which provides the coolant 112 to the tunnel. The coolant 112 is transported through the tube 120 in the handle 28 and to the housing 26 for application onto the skin 22. The handle 28 also includes a light indicator panel. The panel includes lights which are in communication with a microprocessor for providing a status of the apparatus 20 and/or the patient.

In the bipolar example, when the generator is on, supplying the wheels 32 with bipolar current, i.e., positive and negative current, power through the wires 118 is measured repeatedly to determine a rate of heat buildup in the skin 22. As long as the value read by the microprocessor does not go below a predetermined value which is equivalent to the skin 22 being heated with a correlated increase in resistance, a green light 122 is on, directing the user to advance the wheels 32 along and into the skin 22 at a constant speed. When the power in the wire 118 decreases below a set value, which is indicative of the skin 22 resistance being above a maximum allowed temperature buildup, the power to wire 118 is cut-off and a red light 124 is turned on. Finally, when the power is on and the physician rolls the wheels 32 along and into the skin 22 at a pace that does not allow enough time for power to diminish to an optimal value, a yellow light 126 is turned on, cautioning the physician to slow down the pace of rolling the wheels 32 along the skin 22 until the green light 122 is turned on again.

In the monopolar example, when the generator is on, supplying the wheels 32 with positive current, power through the wires 118 is measured repeatedly to determine the rate of heat buildup in the skin 22. The rate of heat buildup is controlled by allowing only two of the wheels 32 to be energized, i.e., fired, at one time and measured repeatedly. As long as the value read by the microprocessor does not go below a set value, equivalent to the skin 22 being heated with the correlated increase in resistance, the green light 122 is on, directing the physician to advance at a constant speed of rolling the wheels 32 along and into the skin 22. When the power decreases and falls below a set value, indicative to skin 22 resistance of a maximum allowed temperature buildup, the power supplied to the wheels 32 is cut off and the red light 124 is illuminated. When the power is on and the physician rolls the electrode wheel at a pace along and into the skin 22 that does not allow the power to diminish to an optimal value, the yellow light 126 is illuminated, directing the physician to slow down until the green light 122 is illuminated once again. In the monopolar application, the total number of wheels 32 which are energized simultaneously, or fired, and the duration of the time which the wheels 32 are fired, is varied. This allows the user to accomplish various treatment modes.

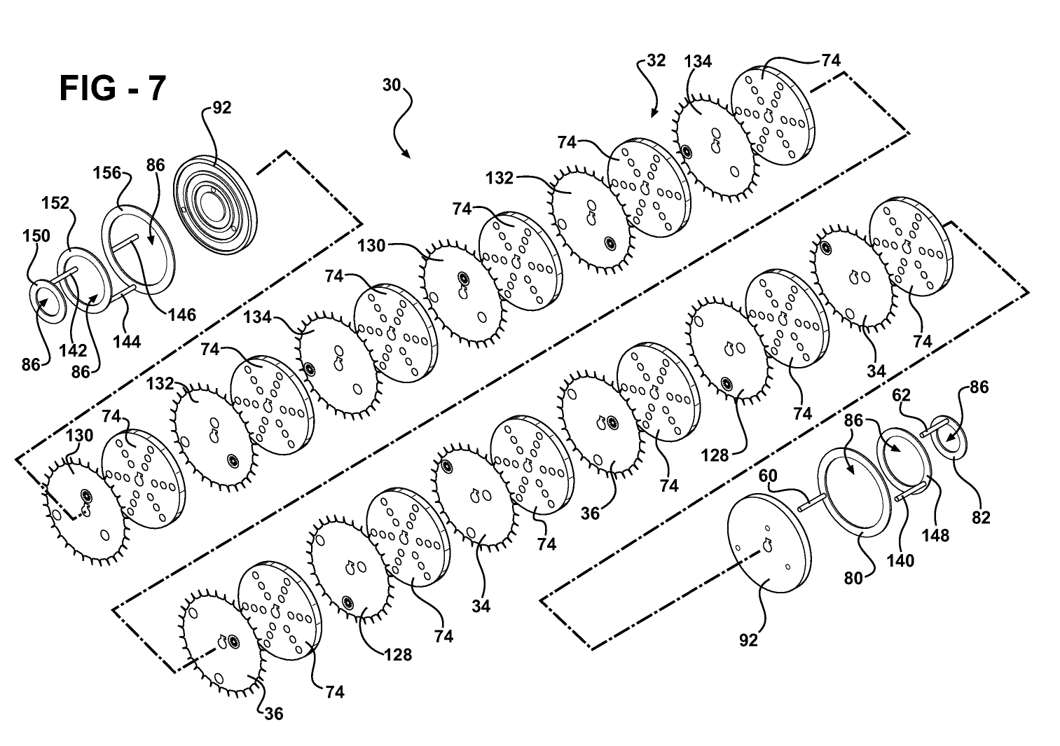

In another example, referring to FIGS. 7-11, the monopolar configuration for the apparatus 20 includes twelve wheels 32 with six different wheel configurations. This means that the first wheels 34 configuration includes two of the first wheels 34, shown in FIG. 4A, and the second wheel 36 configuration includes two of the first wheels 36, shown in FIG. 4B. In addition, a third wheel configuration includes two of a third wheel 128, shown in FIG. 4C. A fourth, fifth, and sixth wheel configuration includes two of a fourth, fifth, and sixth wheel 130, 132, 134, shown generally in FIG. 7. As with the first and second wheels 34, 36, the third, fourth, fifth, and sixth wheels 128, 130, 132, 134 each define the central hole 70 and the respective connector hole 136, bypass hole 138, and connector axis 139. Additionally, a third, fourth, fifth, and six pin 140, 142, 144, 146 each extend through the respective holes 136, 138, along the respective axes 139, to electrically connect the pins 140, 142, 144, 146 to the respective wheels 128, 130, 132, 134 while being electrically insulated from the other wheels 128, 130, 132, 134. A third, fourth, fifth, and sixth contact ring 148, 150, 152, 154 are in electrical communication with the respective third, fourth, fifth, and sixth pin 140, 142, 144, 146. Likewise, a third, fourth, fifth, and sixth contact 156, 158, 160, 162 are disposed on one of the covers 114, 116 to provide and maintain continuous contact with the respective contact rings 148, 150, 152, 154. As discussed above, multiple wheel configurations allow different wheel 32 configurations to be energized at different times to control the temperature of the skin 22.

Many modifications and variations of the present invention are possible in light of the above teachings. In addition, the reference numerals in the claims are merely for convenience and are not to be read in any way as limiting.

What is claimed is:

1. An electrode apparatus for applying electrical current to skin, said apparatus comprising:
a wheel defining a central hole extending along a central axis with said wheel having an outer perimeter;
a plurality of needles disposed about said perimeter and extending radially away from said central axis;
an axle disposed through said central hole along said central axis for allowing said wheel to rotate about said axle on said central axis;
a contact in electrical communication with said wheel to supply electrical current to said needles as said wheel is rolled along the skin.

2. An apparatus as set forth in claim 1 further comprising:
a contact ring defining a ring opening with said axle disposed through said ring opening along said central axis;
said wheel defining a connector hole extending along a connector axis; and
a pin disposed through said connector holes and interconnecting said contact ring and said wheel such that said wheel and said contact ring rotate about said central axis in unison with said contact in electrical communication with said contact ring such that electrical current is supplied to said needles via said wheel and said pin and said contact ring.

3. An apparatus as set forth in claim 2 wherein said contactor is further defined as a ball plunger.

4. An apparatus as set forth in claim 2 further comprising a contact plate disposed on central axis with said contact ring disposed on said contact plate.

5. An apparatus as set forth in claim 2 wherein said wheel is further defined as a plurality of wheels disposed in adjacent relationship along said axis with said axle extending through said center hole along said central axis of each of said wheels and with said pin extending through said connector hole of each of said wheels.

6. An apparatus as set forth in claim 5 wherein said contact ring is further defined as a first and a second contact ring with said first contact ring surrounding said second contact ring about said central axis.

7. An apparatus as set forth in claim 6 wherein said pin is further defined as a first pin and a second pin with said first pin disposed through said first connector holes and in electrical communication with said first contact ring and said second pin disposed through said second connector holes and in electrical communication with said second contact ring.

8. An apparatus as set forth in claim 6 wherein said contact is further defined as a first contact and a second contact with said first contact in electrical communication with each of said first contact ring and said second contact in electrical communication with said second contact ring.

9. An apparatus as set forth in claim 5 wherein each of said plurality of wheels further include a coating of dielectric material.

10. An apparatus as set forth in claim 8 wherein said coating is further defined as substantially covering said wheels.

11. An apparatus as set forth in claim 10 wherein said coating defines a void and said void is defined as an area surrounding one of said first and second connector holes to allow electrical contact between one of said first and second pins and said one of said first and second connector holes and supply electrical current to said needles.

12. An apparatus as set forth in claim 9 wherein said needles are at least partially free of said coating.

13. An apparatus as set forth in claim 1 further comprising a housing extending from said handle with said axle engaged with said housing for at least partially enclosing said wheel and allowing said wheel to rotate with respect to said housing.

14. An apparatus as set forth in claim 13 wherein said housing defines at least one tunnel to transport coolant through said tunnel and onto the skin.

15. An apparatus as set forth in claim 1 wherein said central hole is further defined as having a keyhole shape and said axle is further defined as having a shape which is complimentary to the keyhole shape of said central hole to lock said wheel to said axle and allow said wheel and said axle to rotate in unison.

16. An electrode wheel assembly for applying electrical current to skin, said assembly comprising:
a first and a second plurality of wheels with each of said first and second plurality of wheels having an outer perimeter and with said first and second plurality of wheels disposed in adjacent relationship along a central axis;
a plurality of needles disposed along said perimeter of each of said first and second plurality of wheels with said plurality of needles extending radially away from said central axis;
said first plurality of wheels defining a first connector hole extending along a first connector axis and said second plurality of wheels defining a second connector hole extending along a second connector axis;
said first plurality of wheels defining a first bypass hole extending along said second connector axis and said second plurality of wheels defining a second bypass hole extending along said first connector axis;
a first pin extending along said first connector axis and through said first connector holes and said first bypass holes with said first pin in electrical communication with said first plurality of wheels; and
a second pin extending along said second connector axis and through said second connector holes and said second bypass holes with said second pin in electrical communication with said second plurality of wheels.

17. An assembly as set forth in claim 16 wherein said first and second pins have a rod diameter and said first and second connector holes have a connector diameter and said first and second bypass holes have a bypass diameter with said bypass diameter being greater than said rod diameter and said rod diameter being at least equal to said connector diameter.

18. An assembly as set forth in claim 17 wherein said first pin does not contact said first plurality of wheels as said first pin extends through said first bypass holes and said second pin does not contact said second plurality of wheels as said second pin extends through said second bypass holes.

19. An assembly as set forth in claim 18 wherein said first pin is engaged by said first plurality of wheels at said first rod hole as said first pin extends through said first rod hole and said second pin is engaged by said second plurality of wheels at said second rod hole as said second pin extends through said second rod hole.

20. An assembly as set forth in claim 16 wherein said first pin is electrically insulated from said second plurality of wheels as said first pin extends through said first bypass hole and said second pin is electrically insulated from said first plurality of wheels as said second pin extends through said second bypass hole.

21. An assembly as set forth in claim 16 wherein said first and second plurality of wheels define a central hole extending along said central axis and said assembly further comprises an axle with said axle extending through said central holes along said central axis.

22. An assembly as set forth in claim 16 further comprising a plurality of insulators with one of said insulators disposed between each of said adjacent wheels for electrically insulating each of said first and second plurality of wheels from each other.

23. An assembly as set forth in claim 26 wherein said first plurality of wheels are alternated with said second plurality of wheels along said central axis such that one of said first plurality of wheels are not disposed adjacent one of said second plurality of wheels.

24. An assembly as set forth in claim 16 further comprising a first contact in electrical communication with said first pin and a second contact in electrical communication with said second pin such that said first contact supplies a first electrical current to only said first plurality of wheels and said second contact supplies a second electrical current to only said second plurality of wheels.

25. An assembly as set forth in claim 24 further comprising a first contact ring extending from said first pin and a second contact ring extending from said second pin with said first contact ring in electrical communication with said first contact and rotatable about said central axis with said first and second plurality of wheels and said second contact ring in electrical communication with said second contact.

26. An assembly as set forth in claim 25 further comprising a contact plate disposed along said central axis and adjacent an end of said first and second plurality of wheels with said first contact ring disposed on said contact plate.

27. An assembly as set forth in claim 25 further comprising a first contact having a crest in continuous contact with said first connector ring as said first contact ring rotates about said central axis and a second contact having a crest in continuous contact with said second contact ring as said second contact ring rotates about said central axis.

28. A method of tightening skin using an electrode apparatus including a plurality of wheels rotatable about a central axis with each of the plurality of wheels including a plurality of needles disposed about each of the plurality of wheels, said method comprising the steps of:
placing the electrode apparatus on the skin;
directing an electrical current to the plurality of needles of each of the plurality of wheels such that the needles apply the electrical current to the skin; and
rotating the plurality of wheels along the skin such that at least one of the plurality of needles of the wheels is piercing the skin.

29. A method as set forth in claim 28 wherein said step of directing an electrical current is further defined as the step of directing current from a generator to at least one of the plurality of needles.

30. A method as set forth in claim 29 further comprising the step of attaching an electrical ground to the skin.

31. A method as set forth in claim 28 wherein said step of directing an electrical current is further defined as the step of directing current from a generator to the plurality of wheels and directing current from the other of the plurality of wheels to the generator.

32. A method as set forth in claim 28 further comprising the step of directing a coolant onto the skin to cool the skin.
